(19) United States
(12) Patent Application Publication
(10) Pub. No.: US 2024/0307007 A1
(43) Pub. Date: Sep. 19, 2024

(54) ALARM SYSTEM AND ALARM METHOD FOR MEDICAL EMERGENCY

(71) Applicants: Acer Incorporated, New Taipei City (TW); Far Eastern Memorial Hospital, New Taipei City (TW)

(72) Inventors: Sheng-Wei Chu, New Taipei City (TW); Tsung-Hsien Tsai, New Taipei City (TW); Ke-Han Pan, New Taipei City (TW); Yueh-Yarng Tsai, New Taipei City (TW); Pei-Jung Chen, New Taipei City (TW); Jun-Hong Chen, New Taipei City (TW); Yen-Wen Wu, New Taipei City (TW); Jen-Tang Sun, New Taipei City (TW)

(73) Assignees: Acer Incorporated, New Taipei City (TW); Far Eastern Memorial Hospital, New Taipei City (TW)

(21) Appl. No.: 18/515,283

(22) Filed: Nov. 21, 2023

(30) Foreign Application Priority Data
Mar. 17, 2023 (TW) 112110036

(51) Int. Cl. A61B 5/00; A61B 5/024; A61B 5/11

(52) U.S. Cl. CPC A61B 5/746; A61B 5/0022; A61B 5/02416; A61B 5/1112; A61B 5/7282; A61B 5/7455; A61B 2560/0431

(57) ABSTRACT

An alarm system an alarm method for a medical emergency are provided. The alarm method includes: obtaining a physiological signal of a user by a sensor of a portable electronic device; receiving the physiological signal from the portable electronic device, determining whether an abnormal event has occurred according to the physiological signal, and transmitting first feedback information corresponding to the physiological signal to a server in response to the abnormal event by the terminal device; and outputting an alarm message according to the first feedback information by the server.

### CROSS-REFERENCE TO RELATED APPLICATION

This application claims the priority benefit of Taiwan application serial no. 112110036, filed on Mar. 17, 2023. The entirety of each of the above-mentioned patent applications is hereby incorporated by reference herein and made a part of this specification.

### BACKGROUND

### Technical Field

The invention relates to an e-health technology, and in particular, to an alarm system and an alarm method for medical emergency.

### Description of Related Art

The phenomenon of declining birth rate has led to an increasingly serious global population aging problem, and the manpower used to care for the elderly is also becoming increasingly scarce. The elderly is a high-risk group for cardiovascular diseases, so it is very important to track the cardiovascular status of the elderly in real time. When cardiovascular abnormalities occur in patients with cardiovascular disease, the patient may lose consciousness and be unable to actively seek rescue (for example: press the emergency bell set at a fixed position) or report to the medical institution. Therefore, how to send an alarm message to relevant personnel when an abnormal event occurs is one of the important issues in this field.

### SUMMARY

The present invention provides an alarm system and an alarm method for medical emergency, which can track the user's physiological signals and output an alarm message when an abnormal event occurs.

An alarm system for medical emergency of the present invention includes a portable electronic device, a terminal device, and a server. The portable electronic device includes a sensor, wherein the sensor obtains a physiological signal of a user. The terminal device is communicatively connected to the portable electronic device and receives the physiological signal from the portable electronic device, wherein the terminal device determines whether an abnormal event has occurred according to the physiological signal and transmits a first feedback information corresponding to the physiological signal to a server in response to the abnormal event. The server is communicatively connected to the terminal device and receives the first feedback information from the terminal device, wherein the server outputs an alarm message according to the first feedback information.

In an embodiment of the present invention, the aforementioned sensor includes a photoelectric sensor, and the physiological signal includes a photoplethysmography signal.

In an embodiment of the present invention, the aforementioned terminal device calculates a heart rate of the user according to the photoplethysmography signal, and determines that the abnormal event occurs in response to the heart rate being greater than a first threshold or the heart rate being less than a second threshold.

In an embodiment of the present invention, the aforementioned terminal device calculates a peak-to-peak interval in the photoplethysmography signal, and determines that the abnormal event occurs in response to the peak-to-peak interval being less than a third threshold.

In an embodiment of the present invention, the aforementioned first feedback information includes at least one of the physiological signal and the heart rate, wherein the alarm message is associated with the at least one of the physiological signal and the heart rate.

In an embodiment of the present invention, the aforementioned server displays the at least one of the physiological signal and the heart rate through a graphical user interface to receive user operations, wherein the server outputs the alarm message in response to the user operations.

In an embodiment of the present invention, the aforementioned server outputs the alarm message through at least one of the following: instant messaging software, web browser, email and short message service.

In an embodiment of the present invention, the aforementioned portable electronic device further includes a positioning device. The positioning device detects a position information of the portable electronic device, wherein the terminal device receives the position information from the portable electronic device, wherein the first feedback information includes the position information, and the alarm message includes the position information.

In an embodiment of the present invention, the aforementioned positioning device includes a GPS signal receiver.

In an embodiment of the present invention, the aforementioned terminal device transmits a second feedback information corresponding to the physiological signal to the server according to a preset period, wherein the server outputs a physiological state report of the user according to the second feedback information.

In an embodiment of the present invention, the aforementioned physiological state report includes at least one of the following: a recording period of the physiological signal, a proportion of available physiological signals in the recording period, a proportion of abnormal atrial fibrillation, a frequency of occurrence of atrial fibrillation, a frequency of high heart rate, a frequency of low heart rate, a distribution of the abnormal events, and a waveform diagram of the physiological signal related to the abnormal event.

In an embodiment of the present invention, the aforementioned portable electronic device further includes an actuator. The actuator vibrates in response to the abnormal event.

An alarm method for medical emergency of the present invention, suitable for an alarm system including a portable electronic device, a terminal device, and a server, wherein the alarm method comprises: obtaining a physiological signal of a user by a sensor of a portable electronic device; receiving the physiological signal from the portable electronic device, determining whether an abnormal event has occurred according to the physiological signal, and transmitting a first feedback information corresponding to the physiological signal to a server in response to the abnormal event by the terminal device; and outputting an alarm message according to the first feedback information by the server.

Based on the above, the present invention can monitor the physiological condition of the subject at any time, and can also transmit the physiological signal of the subject to the remote medical personnel for analysis. In this way, the probability of false alarms issued by the alarm system can be significantly reduced.

### BRIEF DESCRIPTION OF THE DRAWINGS

FIG. 1 is a schematic diagram of an alarm system for medical emergency according to an embodiment of the present invention.

FIG. 2 is a flow chart of an alarm method for medical emergency according to an embodiment of the present invention.

### DESCRIPTION OF THE EMBODIMENTS

In order to make the content of the present invention more comprehensible, the following specific embodiments are taken as examples in which the present invention can actually be implemented. In addition, wherever possible, elements/components/steps using the same reference numerals in the drawings and embodiments represent the same or similar parts.

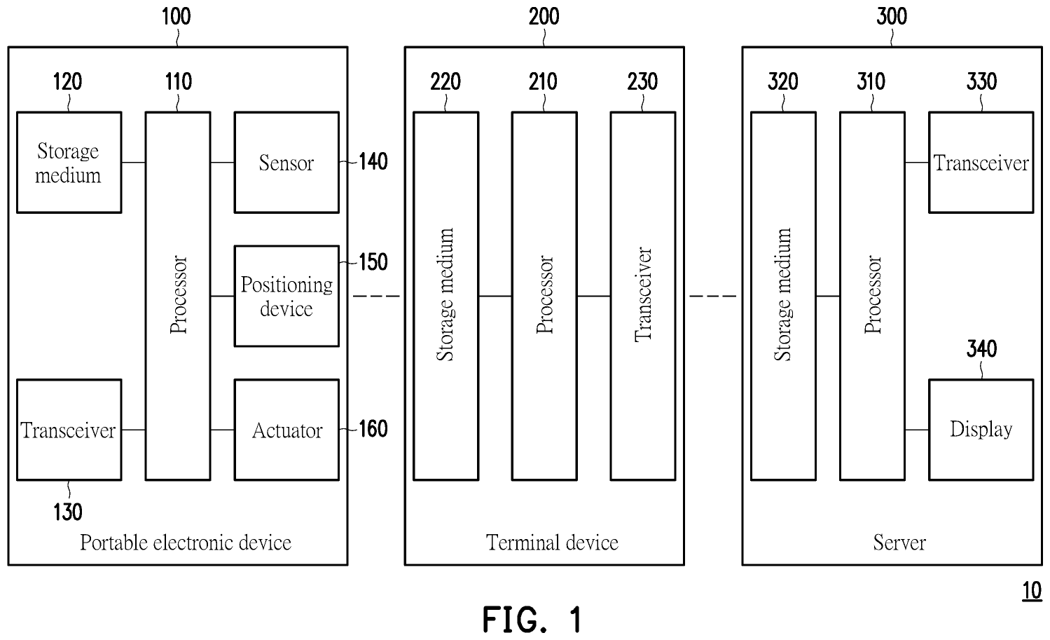

FIG. 1 is a schematic diagram of an alarm system 10 for medical emergency according to an embodiment of the present invention. The alarm system 10 can be used to monitor the user's physiological condition, and output an alarm message to relevant personnel in case of abnormal events, such as the user himself, the user's family members, ambulance personnel or medical personnel. The alarm system 10 can include a portable electronic device 100, a terminal device 200, and a server 300. The terminal device 200 can be communicatively connected to the portable electronic device 100 or the server 300.

The portable electronic device 100 is, for example, a smart bracelet that can be worn by the user. The portable electronic device 100 can include a processor 110, a storage medium 120, a transceiver 130, a sensor 140, a positioning device 150, and an actuator 160.

The processor 110 is, for example, central processing unit (CPU), or other programmable general-purpose or special-purpose micro control unit (MCU), microprocessor, digital signal processor (DSP), programmable controller, application specific integrated circuit (ASIC), graphics processing unit (GPU), image signal processor (ISP), image processing unit (IPU), arithmetic logic unit (ALU), complex programmable logic device (CPLD), field programmable gate array (FPGA) or other similar components or a combination of the above components. The processor 110 can be coupled to the storage medium 120, the transceiver 130, the sensor 140, the positioning device 150 and the actuator 160, and access and execute multiple modules and various applications stored in the storage medium 120.

The storage medium 120 is, for example, any type of fixed or removable random access memory (RAM), read-only memory (ROM), flash memory, hard disk drive (HDD), solid state drive (SSD) or similar components or a combination of the above components, which is used to store a plurality of modules or various application programs executable by the processor 110.

The transceiver 130 transmits and receives signals in a wireless or wired manner. The transceiver 130 can also perform operations such as low noise amplification, impedance matching, frequency mixing, up or down frequency conversion, filtering, amplification, and the like. The portable electronic device 100 can communicate with the terminal device 200 through the transceiver 130.

The sensor 140 is used to measure the physiological signal of the wearer of the portable electronic device 100. The sensor 140 includes, for example, a photoelectric sensor.

The positioning device 150 can be used to detect a position information of the portable electronic device 100, and the position information includes, for example, the longitude, latitude or altitude of the user. The positioning device 150 is, for example, a global positioning system (GPS) signal receiver. The processor 110 can transmit the position information obtained by the positioning device 150 to the terminal device 200 through the transceiver 130.

The actuator 160, for example, includes components that can generate vibrations such as a motor. The actuator 160 is controlled by the processor 110. After the processor 110 determines that an abnormal event occurs, the processor 110 can send a control signal to the actuator 160. The actuator 160 can vibrate in response to receiving the control signal, so as to remind the user wearing the portable electronic device 100 of the occurrence of abnormal events.

The terminal device 200 is, for example, a smart phone or a tablet computer carried by the user. The terminal device 200 can include a processor 210, a storage medium 220, and a transceiver 230.

The processor 210 is, for example, CPU, or other programmable general-purpose or special-purpose MCU, microprocessor, DSP, programmable controller, ASIC, GPU, ISP, IPU, ALU, CPLD, FPGA or other similar components or a combination of the above components. The processor 210 can be coupled to the storage medium 220 and the transceiver 230, and access and execute various modules and various applications stored in the storage medium 220.

The storage medium 220 is, for example, any type of fixed or removable RAM, ROM, a flash memory, HDD, SSD or similar components or a combination of the above components, which is used to store a plurality of modules or various application programs executable by the processor 210.

The transceiver 230 transmits and receives signals in a wireless or wired manner. The transceiver 230 can also perform operations such as low noise amplification, impedance matching, frequency mixing, up or down frequency conversion, filtering, amplification, and the like. The terminal device 200 can communicate with the portable electronic device 100 or the server 300 through the transceiver 230. In an embodiment, the terminal device 200 can communicate with the server 300 through a cloud server, a relay node or the Internet.

The server 300 is, for example, a computer or computing device owned by a medical institution or an ambulance institution. The server 300 can include a processor 310, a storage medium 320, a transceiver 330, and a display 340.

The processor 310 is, for example, CPU, or other programmable general-purpose or special-purpose MCU, microprocessor, DSP, programmable controller, ASIC, GPU, ISP, IPU, ALU, CPLD, FPGA or other similar components or a combination of the above components. The processor 21 can be coupled to the storage medium 320, the transceiver 330, and the display 340, and access and execute various modules and various applications stored in the storage medium 320.

The storage medium 320 is, for example, any type of fixed or removable RAM, ROM, a flash memory, HDD, SSD or similar components or a combination of the above components, which is used to store a plurality of modules or various application programs executable by the processor 310.

The transceiver 330 transmits and receives signals in a wireless or wired manner. The transceiver 330 can also perform operations such as low noise amplification, impedance matching, frequency mixing, up or down frequency conversion, filtering, amplification, and the like. The server 300 can communicate with the terminal device 200 through the transceiver 330. In an embodiment, the server 300 can communicate with the terminal device 200 through a cloud server, a relay node or the Internet.

The display 340 can be liquid-crystal display (LCD), light-emitting diode (LED) display, vacuum fluorescent display (VFD), plasma display panel (PDP), organic light-emitting diode (OLED), or field-emission display (FED).

The portable electronic device 100 can obtain a physiological signal of the user through the sensor 140, and can transmit the physiological signal or the position information to the terminal device 200 through the transceiver 130. In an embodiment, the sensor 140 may include a photoelectric sensor, and the physiological signal may include a photoplethysmography (PPG) signal.

After receiving the physiological signal, the processor 210 of the terminal device 200 can extract a feature value from the physiological signal. In an embodiment, the processor 210 can calculate the user's heart rate according to the PPG signal, wherein the heart rate is, for example, the user's average heart rate during a preset period of time (for example: 32 seconds). In an embodiment, the processor 210 can calculate a peak-to-peak interval (PPI) in the PPG signal, where the peak-to-peak interval is, for example, the average peak-to-peak interval of the user during a predetermined period of time (such as, 32 seconds).

After obtaining the feature value of the physiological signal (for example: heart rate or peak-to-peak interval), the processor 210 can determine whether an abnormal event related to the user's cardiovascular system occurs according to the feature value. In an embodiment, the processor 210 may determine that an abnormal event occurs in response to the user's heart rate being greater than a first threshold (for example: 170 bpm) or less than a second threshold (for example: 40 bpm, wherein the second threshold is less than the first threshold). In an embodiment, the processor 210 may determine that an abnormal event occurs in response to the user's peak-to-peak interval being less than a third threshold.

If the processor 210 determines that an abnormal event occurs, the processor 210 may transmit a first feedback information corresponding to the physiological signal to the server 300, the first feedback information may include information such as the physiological signal, the feature value of the physiological signal (such as heart rate or peak-to-peak interval), or the position information measured by the positioning device 150. If the processor 210 determines that no abnormal event has occurred, the processor 210 may not transmit the first feedback information to the server 300, so as to save power or communication resources consumed by the terminal device 200.

After receiving the first feedback information, the processor 310 of the server 300 can generate and output the alarm message according to the first feedback information, wherein the alarm message can be related to the physiological signal, the feature value of the physiological signal (such as heart rate or peak-to-peak interval), or the position information and other information. The processor 310 can, for example, transmit the alarm message to relevant personnel through instant messaging software, web browser, email, or short message service (SMS), etc.

In an embodiment, before the server 300 outputs the alarm message, the server 300 can display a graphical user interface through the display 340 for medical personnel to watch, wherein the graphical user interface may include the physiological signal, the feature value of the physiological signal (such as heart rate or peak-to-peak interval), or the position information and other information. The medical personnel can determine whether the abnormal event occurred this time is a misjudgment according to the physiological signal or the feature value. If the medical personnel determines that the abnormal event is not a misjudgment, the medical personnel can operate the server 300. The server 300 can receive user operations from the medical personnel, and output the alarm message according to the user operations. That is to say, the present invention provides a rejudgment mechanism for the medical personnel on abnormal events, so as to avoid the waste of medical information or rescue information caused by misjudgment of abnormal events.

In an embodiment, the processor 210 can transmit a second feedback information corresponding to the physiological signal to the server 300 according to a preset period. Compared with the first feedback information which is transmitted to the server 300 in real time when an abnormal event occurs, the second feedback information can contain long-term information. For example, the processor 210 can transmit the second feedback information related to the user's physiological signals throughout the day to the server 300 at a preset period of one day.

The processor 310 of the server 300 can generate and output a physiological state report of the user according to the second feedback information. For example, the processor 310 can display the physiological state report through the display 340 for medical personnel or ambulance personnel to watch. The physiological state report may include but not limited to a recording period of the physiological signal, a proportion of available physiological signals in the recording period, a proportion of abnormal atrial fibrillation, a frequency of occurrence of atrial fibrillation, a frequency of high heart rate, a frequency of low heart rate, a distribution of the abnormal events, and a waveform diagram of the physiological signal related to the abnormal event and other information, wherein the aforementioned information can be transmitted from the terminal device 200 to the server 300 through the second feedback information.

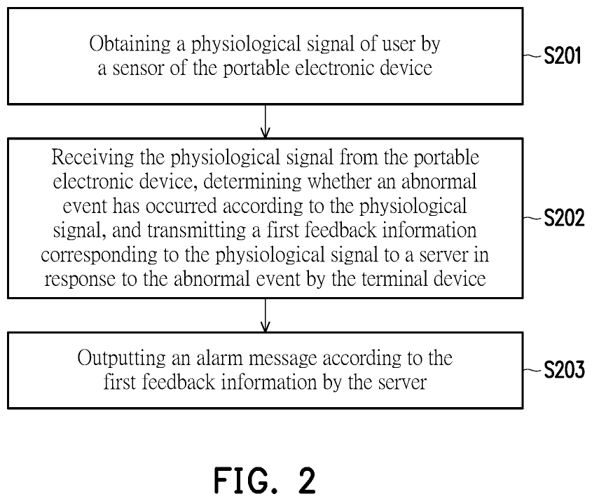

FIG. 2 is a flow chart of an alarm method for medical emergency according to an embodiment of the present invention, wherein the alarm method can be implemented by the alarm system 10 shown in FIG. 1. In step S201, the physiological signal of user is obtained by the sensor of the portable electronic device. In step S202, the physiological signal is received from the portable electronic device, determined whether an abnormal event has occurred according to the physiological signal, and the first feedback information corresponding to the physiological signal is transmitted to a server in response to the abnormal event by the terminal device. In step S203, the alarm message is outputted according to the first feedback information by the server.

In summary, the portable electronic device of the present invention can be worn by the subject to measure the physiological signal of the subject in real time, and transmit the physiological signal to the terminal device with computing power. The terminal device can monitor the physiological signal to determine whether the subject has abnormal events associated with cardiovascular disease. The terminal device can transmit the relevant feedback information of the subject to the medical personnel or ambulance personnel when an abnormal event occurs, so as to report the physiological state and location information of the subject. After the medical personnel analyze the feedback information and determine that the report of the abnormal event is not a false alarm, the server can output the alarm message to the relevant personnel according to the user operation of the medical personnel. The present invention can not only monitor the physiological condition of the subject at any time, but also transmit the physiological signal of the subject to remote medical personnel for analysis. In this way, the probability of false alarms can be significantly reduced. Compared with the emergency equipment set in a fixed position (for example: emergency bell), the present invention can automatically report abnormal events through the portable electronic device that is easy to wear and small in size and the terminal device that does not require additional cost, so as to prevent the subject from losing consciousness and being unable to actively seek assistance.

What is claimed is:

1. An alarm system for medical emergency, comprising:
a portable electronic device, including a sensor, wherein the sensor obtains a physiological signal of a user;
a terminal device, communicatively connected to the portable electronic device and receiving the physiological signal from the portable electronic device, wherein the terminal device determines whether an abnormal event has occurred according to the physiological signal and transmits a first feedback information corresponding to the physiological signal to a server in response to the abnormal event; and
a server, communicatively connected to the terminal device and receiving the first feedback information from the terminal device, wherein the server outputs an alarm message according to the first feedback information.

2. The alarm system according to claim 1, wherein the sensor comprises a photoelectric sensor, and the physiological signal comprises a photoplethysmography signal.

3. The alarm system according to claim 2, wherein the terminal device calculates a heart rate of the user according to the photoplethysmography signal, and determines that the abnormal event occurs in response to the heart rate being greater than a first threshold or the heart rate being less than a second threshold.

4. The alarm system according to claim 2, wherein the terminal device calculates a peak-to-peak interval in the photoplethysmography signal, and determines that the abnormal event occurs in response to the peak-to-peak interval being less than a third threshold.

5. The alarm system according to claim 3, wherein the first feedback information comprises at least one of the physiological signal and the heart rate, wherein the alarm message is associated with the at least one of the physiological signal and the heart rate.

6. The alarm system according to claim 5, wherein the server displays the at least one of the physiological signal and the heart rate through a graphical user interface to receive user operations, wherein the server outputs the alarm message in response to the user operations.

7. The alarm system according to claim 1, wherein the server outputs the alarm message through at least one of the following: instant messaging software, web browser, email and short message service.

8. The alarm system according to claim 1, wherein the portable electronic device further comprises:
a positioning device, detecting a position information of the portable electronic device, wherein the terminal device receives the position information from the portable electronic device, wherein the first feedback information comprises the position information, and the alarm message comprises the position information.

9. The alarm system according to claim 8, wherein the positioning device comprises a GPS signal receiver.

10. The alarm system according to claim 1, wherein the terminal device transmits a second feedback information corresponding to the physiological signal to the server according to a preset period, wherein the server outputs a physiological state report of the user according to the second feedback information.

11. The alarm system according to claim 10, wherein the physiological state report comprises at least one of the following: a recording period of the physiological signal, a proportion of available physiological signals in the recording period, a proportion of abnormal atrial fibrillation, a frequency of occurrence of atrial fibrillation, a frequency of high heart rate, a frequency of low heart rate, a distribution of the abnormal events, and a waveform diagram of the physiological signal related to the abnormal event.

12. The alarm system according to claim 1, wherein the portable electronic device further comprises:
an actuator, vibrating in response to the abnormal event.

13. An alarm method for medical emergency, suitable for an alarm system including a portable electronic device, a terminal device, and a server, wherein the alarm method comprises:
obtaining a physiological signal of a user by a sensor of the portable electronic device;
receiving the physiological signal from the portable electronic device, determining whether an abnormal event has occurred according to the physiological signal, and transmitting a first feedback information corresponding to the physiological signal to a server in response to the abnormal event by the terminal device; and
outputting an alarm message according to the first feedback information by the server.
